(19) United States
(12) Patent Application Publication
(10) Pub. No.: US 2015/0037379 A1
(43) Pub. Date: Feb. 5, 2015

(54) COSMETIC COMPOSITIONS

(71) Applicant: Christine Mendrok-Edinger, Basel (CH)

(72) Inventor: Christine Mendrok-Edinger, Basel (CH)

(73) Assignee: DSM IP ASSETS B.V., Heerlen (NL)

(21) Appl. No.: 14/124,744

(22) PCT Filed: Jun. 6, 2012
(86) PCT No.: PCT/EP2012/060669

§ 371 Date: Mar. 14, 2014

(30) Foreign Application Priority Data
Jun. 8, 2011 (EP) 11169201.8
Jun. 15, 2011 (EP) 11169916.1

(51) Int. Cl. A61K 8/49; A61Q 17/04; A61K 8/29; A61K 8/02; A61K 8/55; A61K 8/37

(52) U.S. Cl. CPC A61K 8/496; A61K 8/55; A61K 8/37; A61K 8/29; A61K 8/0241; A61Q 17/04; A61K 2800/42
USPC 424/401; 424/59

(57) ABSTRACT

The present invention relates to a composition for topical application comprising in a physiologically acceptable medium at least a benzotriazol derivative, a microfine metal oxide particle, a cosmetic solvent selected from the group of benzoate solvents such as phenethyl benzoate and a phosphate ester emulsifier. More specifically, the invention relates to compositions that exhibit an increased whitening effect on the skin.

The present invention relates to a composition for topical application comprising in a physiologically acceptable medium at least a benzotriazol derivative, a microfine metal oxide particle, a cosmetic solvent selected from the group of benzoate solvents such as phenethyl benzoate and a phosphate ester emulsifier. More specifically, the invention relates to compositions that exhibit an increased whitening effect on the skin.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

In addition to chemical UV-filter substances, the use of physical UV-filter substances is known in the art. Such physical UV-filter substances are e.g. microfine zinc oxide and microfine titanium dioxide which are suspended in the respective a cream or lotion and which are highly appreciated because of their excellent safety profile. Furthermore, such physical UV-filter tend to leave a white layer on the skin which is highly appreciated by Asians. However, the white layer generated is often not sufficient to lead to the desired whitening effect of the skin.

It was therefore the object of the present invention to remedy the disadvantages of the prior art and to develop sun care products comprising microfine metal oxide particles which exhibit an increased skin whitening effect.

Surprisingly, it has been found that the use of a phosphate ester emulsifier in the presence of specific benzotriazol derivatives and a cosmetic solvent selected from the group of benzoate solvents such as phenethyl benzoate increases the whitening effect of the microfine metal oxide particles.

Thus, the invention relates in one aspect to a topical composition comprising a phosphate ester emulsifier, a cosmetic solvent selected from the group of benzoate solvents such as particularly phenethyl benzoate, a microfine metal oxide particle and at least one benzotriazol derivative of formula (I)

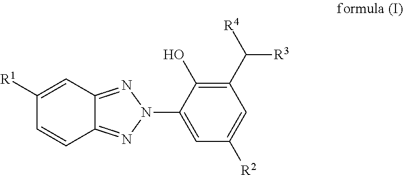

wherein

R¹ is hydrogen; C₁₋₅alkyl; C₁₋₅alkoxy or halogen; preferably hydrogen or chloride; most preferably hydrogen;

R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;

R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and

R⁴ is hydrogen or C₁₋₅alkyl; preferably hydrogen.

In another aspect the invention relates to the use a phosphate ester emulsifier, a cosmetic solvent selected from the group of benzoate solvents such as particularly phenethyl benzoate and a benzotriazol derivative of formula (I) to increase the whitening effect of microfine metal oxide particles.

In yet a further embodiment the invention relates to a method for increasing the whitening effect of microfine metal oxide particles incorporated into a topical composition said method comprising the addition of a phosphate ester emulsifier, a cosmetic solvent selected from the group of benzoate solvents such as particularly phenethyl benzoate and at least one benzotriazol of formula (I) to said composition and observing or appreciating the result (e.g. on human skin or another substrate).

The term “Cₓ-C_{y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl pentyl, 3-methylpentyl, 4-methylpentyl, 1,1-dimethylpropyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyle, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl, in particular C₁₋₅alkyl (mono- or poly-)substituted cyclic, bicyclic or tricyclic hydrocarbon residues such as in particular cyclopentyl, cyclohexyl, cycloheptyl or decahydronaphtyl residues. Preferably, C₅₋₁₀cycloalkyl denotes to unsubstituted or C₁₋₂alkyl (mono- or poly-)substituted cyclopentyl, cyclohexyl or cycloheptyl such as in particular to unsubstituted or methyl (mono- or poly-)substituted cyclohexyl such as most in particular cyclohexyl or 3,3,5-trimethyl-cyclohexyl.

The term “Cₓ-C_{y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term microfine metal oxide particle as used herein refers to microfine metal oxide particle suitable as inorganic UV-filter substances. Suitable metal oxides are for example titanium oxide (amorphous or crystallized in rutile and/or anatase form), iron oxide, zinc oxide, zirconium oxide or cerium oxide. For the purposes of the present invention, preferred metal oxides to be mentioned are titanium dioxide and zinc oxide, particularly titanium dioxide.

The term “microfine”, refers to a particle size from 1 nm to 500 nm, particularly from 10 nm to 200 nm or even 15 to 100 nm. The particles may also be coated. Such coatings are well known in the art.

Particularly preferred according to the present invention are microfine titanium dioxide particles. The particle size of the titanium dioxide is not particularly limited. All particle sizes which are principally useful for incorporating into sunscreen compositions can be used in topical compositions according to the present invention. However, the primary particle size of the titanium dioxide is usually in the range from 2 to 100 nm, preferably in the range of 5 to 50 nm and the secondary particle size is preferably between 0.05 and 50 μm, preferably between 0.1 and 1 μm.

The microfine metal oxide particle may be uncoated or may be provided with a coating.

The term “coated” or “coating” means, that the metal oxide particles have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal (e.g. titanium, silicone or aluminum) alkoxides, polyethylene, silicones, proteins (collagen or elastin), alkanolamines, silicon oxides, metal oxides, sodium hexametaphosphate, alumina or glycerol. Such a surface treatment results in a coating of the metal oxide particle.

In a preferred embodiment, the metal oxide particles such as particularly the titanium dioxide are coated. Preferably the coating of the metal oxide particles comprises a silicone containing polymer and/or an inorganic oxide. More preferably, the coating comprises a silicone containing polymer, or an inorganic oxide and a silicone containing polymer. Especially preferred is a coating, which comprises an inorganic oxide and a silicone containing polymer.

The term “silicon containing polymer” refers to synthetic polymeric compounds comprising silicon atoms, which are linked via oxygen atoms to polymers and wherein the residual valences of the silicon atoms are saturated by hydrogen and/or organic residues. Examples of silicon containing polymers are silicones (also referred to as silicon oils) such as methicone, dimethicone, simethicone, polysilicone-15 or a copolymer of methicone and dimethicone.

The term “inorganic oxide” refers to oxides and hydroxides of inorganic elements, such as of silicon (e.g. silica, SiO₂), aluminum (e.g. alumina Al₂O₃, or aluminum hydroxide Al(OH)₃), zirconium or iron, preferably aluminum and silicon. Preferred inorganic oxides are alumina, aluminum hydroxide and silicon dioxide (also known as silica).

In one embodiment, the coating of the metal oxide particles comprises a silicon containing polymer and an inorganic oxide. Suitable examples include, but are not limited to coated titanium oxide particles, which were surface-treated with silica, alumina and silicone oil (such as “Micro Titanium Dioxide MT 100 SAS”, “Micro Titanium Dioxide MT 600 SAS” and “Micro Titanium Dioxide MT 500 SAS” from Tayca) or alumina and silicon (such as “UVT-M262” from Kemira). Further examples of coated titanium dioxide particles are available from BASF as T-Lite SF (titanium dioxide coated with aluminum hydroxide and dimethicone/methicone copolymer; titanium dioxide content 79-89 wt %), T-Lite SF-S (titanium dioxide coated with hydrated silica, dimethicone/methicone copolymer and aluminum hydroxide; titanium dioxide content 73-83 wt %) or T-lite MAX (titanium dioxide coated with dimethoxydiphenylsilane/triethoxycaprylylsilane crosspolymer, hydrated silica and aluminum hydroxide; titanium dioxide content 69-73 wt %). A further suitable coated titanium dioxide (Silica/dimethicone) is sold as PARSOL TX by DSM Nutritional Products Ltd Kaiseraugst.

Examples of coated zinc oxide particles encompass those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane), or those sold under the name “Fuji ZNO-SMS-10” by the company Fuji Pigment (ZnO coated with silica and polymethylsilsesquioxane).

In another embodiment, the coating of the metal oxide particles comprises at least one silicon containing polymer. Suitable examples include, but are not limited to titanium oxides particles treated with octylsilane (such as AEROXIDE® TiO₂ T 805 by Degussa) or with trimethoxycaprylsilane (such as Uvinul® TiO₂ by BASF SE Ludwigshafen).

Examples of zinc oxide particles coated with at least one silicon containing polymer are sold under the name “Zinc Oxide CS-5” by the company Toshibi (ZnO coated with polymethylhydrogenosiloxane), “SPD-ZI” by the company Shin-Etsu (ZnO coated with silicon-grafted acrylic polymer, dispersed in cyclodimethylsiloxane). Further coated zinc oxide particles are commercially available from BASF SE as Z-COTE HP1 (98 wt % zinc oxide and 2 wt % triethoxycaprylylsilane) or Z-COTE MAX (96-99 wt % zinc oxide and 1-4 wt % dimethoxydiphenylsilane/triethoxycaprylylsilane crosspolymer).

In another embodiment, the coating of the metal oxide particles comprises at least one inorganic oxide. Suitable examples include, but are not limited to titanium oxide particles, which were surface-treated with silica and alumina (such as “Micro Titanium Dioxide MT 500 SA” and “Micro Titanium dioxide MT 100 SA” from Tayca, and “Tioveil Fin”, “Tioveil OP”, “Tioveil MOTG” and “Tioveil IPM” from Tioxide), alumina and aluminum stearate (such as “Micro Titanium Dioxide MT 100 T” from Tayca), alumina and aluminum laurate (such as “Micro Titanium Dioxide MT 100 S” from Tayca), iron oxides and iron stearate (such as “Micro Titanium Dioxide MT 100 F” from Tayca), alumina and stearic acid (such as the product “UVT-M160” from Kemira), alumina and glycerol (such as the product “UVT-M212” from Kemira) and silicon dioxide (such as e.g. Eusolex® T-Avo and Eusolex® T-Oleo from Merck/Sachtleben).

The microfine metal oxide particles may have undergone one or at least two surface treatments.

Preferably, the microfine metal oxide particles have undergone at least two surface treatments. Thus, the particles may have a single or more coatings (muilticoating). Preferably, the microfine metal oxide particles have a multicoating such as at least a double coating. The coating may be a multicoating of an inorganic oxide such as preferably silicon dioxide (silica), and a silicon containing polymer, preferably dimethicone.

The terms “methicone [CAS 9004-73-3]” “dimethicone [CAS 63148-62-9]” and “simethicone [CAS 8050-81-5]” stand for methyl hydrogen polysiloxane, dimethyl polysiloxane, respectively, a mixture of poly(dimethylsiloxane) and silicon dioxide.

Particularly preferred microfine metal oxide particles according to the present invention are coated titanium dioxide particles in which the particles are coated by a first layer of an inorganic oxide such as preferably silica and an outer layer of an organic coating such as a silicon containing polymer (such as e.g. methicone, dimethicone or polysilicone-15), stearic acid and glycerol as well as mixtures thereof. Preferably, the inner coating is silica and the outer coating is methicone (such as e.g. the product commercialized as UV-Titan X195 by Kemira) or dimethicone (such as e.g. the product commercialized as PARSOL® TX by DSM Nutritional Products Ltd Kaiseraugst).

The term phosphate esters emulsifier refers to compounds of formula (II)

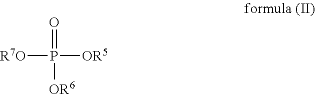

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. Particular advantageous phosphate ester emulsifiers according to the invention are cetyl phosphates such as in particular potassium cetyl phosphate which is e.g. commercially available as Amphisol® K at DSM Nutritional Products Ltd Kaiseraugst.

The term “benzoate solvent” refers in particular to Behenyl Benzoate (Finsolv 137), Benzyl Benzoate (Morflex Benzyl Benzoate), Butyloctyl Benzoate, C12-15 Alkyl Benzoate (Finsolv TN), C16-17 Alkyl Benzoate (Finsolv G2), Castor Oil Benzoate (Finsolv BCO-115), Cetyl Ricinoleate Benzoate (Finsolv BCR-111), C12-15 Pareth-3 Benzoate (Dermol Z5-3B), Dipentaerythrityl Hexabehenate/Benzoate/Ethylhexanoate (Nomcort HR-822), Dipropylene Glycol Dibenzoate (Finsolv PG-22, Uniflex 9-88), Ethylhexyl Benzoate (Finsolv EB), Ethylhexyl Hydroxystearate Benzoate (Finsolv BOHS-111), Hexyldecyl benzoate(kein Hersteller), Isobutyl Benzoate (kein Hersteller), Isostearyl Benzoate (Finsolv SB), laureth-2 Benzoate (Bernel Ester 126), Lauryl/Myristyl Benzoate (Corum 5014), Linalyl Benzoate (Linalyl Benzoate), Octyldodecyl Benzoate (Finsolv BOD), Oleth-2 Benzoate (kein Hersteller), Panthenyl Ethyl Ether Benzoate, Pentaerythrityl Tetrabehenate/Benzoate/Ethyl hexanoate (Salacos P-B822), Pentaerythrityl Tetrabenzoate (Uniplex 552), Pentaerythrityl Tetraethylhexanoate/Benzoate (Salacos BO-63, Salacos P-B8(75)), Phenethyl Benzoate (X-Tend 226, Finsolv Sun), Phenyl Benzoate, PPG-15 Stearyl Ether Benzoate (insolv P), Propyl Benzoate, Propylene Glycol Dibenoate (LexFeel Shine), Stearyl Benzoate (Finsolv 116).

Phenethyl benzoate [INCI Name] is also known as benzoic acid, 2-phenyl ethyl ester [CAS No. 94-47-3] and is e.g. available as X-Tend™226 at International Specialty Products (I.S.P.).

The amount of the microfine metal oxide particle in the topical compositions according to the present invention is advantageously selected in the range of 0.5 to 50 wt.-% such as in particular in the range of 1 to 25 wt.-%, such as most in particular in the range of 1 to 10 wt.-%, based on the total weight of the composition.

The amount of the at least one benzotriazol derivative of formula (I) in the compositions according to the invention is preferable selected in the range of 1 to 20 wt.-%, such as in the range of 2 to 15 wt.-%, in particular in the range of 4 to 10 wt.-%, and most particular in the range of 4 to 8 wt.-%, based on the total weight of the composition.

The cosmetic solvent selected from the group of benzoate solvents such as particularly phenethyl benzoate is preferably used in an amount of 0.5 to 20 wt.-% such as in particular in the range of 1 to 10 wt.-% such as most in particular in the range of 1 to 6 wt.-%, based on the total weight of the composition.

The phosphate ester emulsifier is preferably used in an amount of 0.5 to 10 wt.-% such as in particular in the range of 0.5 to 5 wt.-% such as most in particular in the range of 1 to 4 wt.-% based on the total weight of the composition.

In a particular embodiment of the present invention the benzotriazol derivative is selected from compounds of formula (I) wherein R¹ and R⁴ are hydrogen, R² is methyl and R³ is C₆₋₁₀alkoxy such as preferably C₆₋₁₀alkoxy, or C₆cycloalkoxy, such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124).

In another particular embodiment of the present invention the compound of formula (I) is a compound wherein R¹ and R⁴ are hydrogen, R² is methyl and R³ is undecyl (C₁₁H₂₃) which is commercially available as Tinogard® TL [INCI Name: benzotriazolyl dodecyl p-cresol; IUPAC Name, 2-(2H-benzotriazol-2-yl)-6-dodecyl-4-methyl-phenol] at BASF SE Ludwigshafen.

In another embodiment, the invention relates to topical compositions according to the present invention wherein the compound of formula (I) is benzotriazolyl dodecyl p-cresol, the microfine metal oxide is a double coated titanium dioxide having an inner silica coating and an outer coating selected from the group of methicone and/or dimethicone and the phosphate ester emulsifier is a cetyl phosphate such as in particular potassium cetyl phosphate.

In another particular embodiment, the invention relates to topical compositions according to the present wherein the compound of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the microfine metal oxide is a double coated titanium dioxide having an inner silica coating and an outer coating selected from the group of methicone and/or dimethicone and the phosphate ester emulsifier is a cetyl phosphate such as in particular potassium cetyl phosphate.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

The topical compositions according to the present invention furthermore contain advantageously at least one co-surfactant. Co-surfactants, which are amphiphilic, relatively short chain molecules, are not suitable to form the emulsions, as they are not suitable to form micelles. However, the co-surfactants are able to fill up holes left by the emulsifier and thus further stabilize the emulsion.

Suitable co-surfactants encompass mono- and diglycerides and/or fatty alcohols. The co-surfactant is advantageously used in an amount selected in the range of 0.1 to 10 wt.-% such as in the range of 0.5 to 5 wt.-% such as in particular in the range of 1 to 3 wt.-% based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) and mixtures thereof.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

Suitable UV-filter substances may be organic or inorganic compounds. Exemplary organic UV-filter substances encompass e.g. acrylates such as e.g. 2-ethylhexyl 2-cyano-3,3-diphenylacrylate (octocrylene, PARSOL® 340), ethyl 2-cyano-3,3-diphenylacrylate; Camphor derivatives such as e.g. 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, terephthalylidene dicamphor sulfonic acid (Mexoryl® SX); Cinnamate derivatives such as e.g. ethylhexyl methoxycinnamate (PARSOL® MCX), ethoxyethyl methoxycinnamate, isoamyl methoxycinnamate as well as cinnamic acid derivatives bond to siloxanes; p-Aminobenzoic acid derivatives such as e.g. p-aminobenzoic acid, 2-ethylhexyl p-dimethylaminobenzoate, N-oxypropylenated ethyl p-aminobenzoate, glyceryl p-aminobenzoate; Benzophenones such as e.g. benzophenone-3, benzophenone-4, 2,2′,4,4′-tetrahydroxy-benzophenone, 2,2′-dihydroxy-4,4′-dimethoxybenzophenone; Esters of benzalmalonic acid such as e.g. di-(2-ethylhexyl) 4-methoxybenzalmalonate; Organosiloxane compounds carrying chromophore groups such as e.g. polysilicones-15 (PARSOL® SLX), drometrizole trisiloxane (Mexoryl® XL); Imidazole derivatives such as e.g. 2-phenyl benzimidazole sulfonic acid (PARSOL®HS) and salts thereof such as e.g. sodium- or potassium salts, ammonium salts, morpholine salts, salts of primary, sec. and tert. amines like monoethanolamine salts, diethanolamine salts; Salicylate derivatives such as e.g. isopropylbenzyl salicylate, benzyl salicylate, butyl salicylate, ethylhexyl salicylate (PARSOL® EHS, Neo Heliopan® OS), isooctyl salicylate or homomethyl salicylate (homosalate, PARSOL® HMS, Neo Heliopan® HMS); Triazine derivatives such as e.g. ethylhexyl triazone (Uvinul® T-150), diethylhexyl butamido triazone (Uvasorb® HEB), bis-ethylhexyloxyphenol methoxyphenyl triazine (Tinosorb® S); Benzotriazole derivatives such as e.g. 2,2′-methylene-bis-(6-(2H-benzotriazole-2-yl)-4-(1,1,3,3,-tetramethylbutyl)-phenol (Tinosorb® M); Encapsulated UV-filters such as e.g. encapsulated ethylhexyl methoxycinnamate (Eusolex® UV-pearls) or microcapsules loaded with UV-filters as e.g. dislosed in EP 1471995; Phenylene-1,4-bis-benzimidazolsulfonic acids or salts such as e.g. 2,2-(1,4-phenylene)bis-(1H-benzimidazol-4,6-disulfonic acid) (Neoheliopan AP); Benzoxazol-derivatives such as e.g. 2,4-bis-[5-1(dimethylpropyl)benzoxazol-2-yl-(4-phenyl)-imino]-6-(2-ethylhexyl)-imino-1,3,5-triazin [Uvasorb® K2A); Dibenzoylmethane derivatives such as e.g. Butyl Methoxy Dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd and amino substituted benzophenones such as Diethylamino hydroxybenzoyl hexyl benzoate [IUPAC Name: Hexyl 2-[4-(diethylamino)-2-hydroxybenzoyl]benzoate] sold under the tradename Uvinul® A plus by BASF. Inorganic UV-filter substances encompass pigments such as e.g. microparticulated Zink oxide or Titanium dioxide (e.g. commercially available as PARSOL® TX) The term “microparticulated” refers to a particle size from about 5 nm to about 200 nm, particularly from about 15 nm to about 100 nm. The particles may also be coated by other metal oxides such as e.g. aluminum or zirconium oxides or by silica or by organic coatings such as e.g. polyols, methicone, aluminum stearate, alkyl silane. Such coatings are well known in the art.

In order to enhance the photostability of sun care products it may be desirable to add a photostabilizer. Exemplary photostabilizers known to a skilled person in the art encompass e.g. 3,3-diphenylacrylate derivatives such as e.g. octocrylene (PARSOL® 340) or Polyester-8 (Polycrylene®) or Methoxycrylene (Solastay S1®); Benzylidene camphor derivatives such as e.g. 4-methyl benzylidene camphor (PARSOL® 5000); Benzalmalonate derivatives such as e.g. polysilicones-15 (PARSOL® SLX) or diethylhexyl syringylidene malonate (Oxynex ST liquid); Dialkyl naphthalates such as diethylhexyl naphthalate (Corapan TQ) without being limited thereto. An overview on further stabilizers is e.g. given in ‘SPF Boosters & Photostability of Ultraviolet Filters’, HAPPI, October 2007, p. 77-83 which is included herein by reference. The photostabilizers are generally used in an amount of 0.05 to 10 wt.-% with respect to the total weigh of the topical composition.

Generally, the amount of each UV-filter substance in the compositions according to the invention is selected in the range of about 0.1 to 10 wt.-%, preferably in the range of about 0.2 to 7 wt.-%, most preferably in the range of about 0.5 to 5 wt.-% with respect to the total weigh of the topical composition.

The total amount of UV-filter substances in the compositions according to the invention is preferably in the range of about 1 to 40 wt.-%, preferably in the range of about 5 to 30 wt.-%, in particular in the range of 10 to 30 wt.-% with respect to the total weight of the topical composition.

Preferred UVB-filter substances according to the invention encompass polysilicones-15, phenylbenzimidazol sulfonic acid, octocrylene, ethylhexyl methoxycinnamate, ethylhexyl triazone, ethyl hexylsalicylate, 4-methyl benzylidene camphor, benzophenones-3 and/or homosalate.

Preferred broadband UV-filter substances according to the invention encompass unsymmetrical s-triazine derivatives such 2,4-Bis-{[4-(2-ethyl-hexyloxy)-2-hydroxy]-phenyl}-6-(4-methoxyphenyl)-1,3,5-triazin, certain benzophenones such as e.g. 2-Hydroxy-4-methoxy-benzophenon, 2,2′-Methylen-bis-(6-(2H-benzotriazol-2-yl)-4-(1,1,3,3-tetramethyl-butyl)-phenol), and/or titanium dioxide.

Preferred UVA-filter substances encompass Butyl Methoxy Dibenzoylmethane and Diethylamino hydroxybenzoyl hexyl benzoate.

It is particularly suitable, if the topical compositions according to the present invention are sun care preparations comprising at least one, preferably at least two further UV-filter substances. Preferably the additional UV filter substances are selected from the group consisting of butyl methoxydibenzoylmethane, octocrylene, 4-methylbenzylidene camphor and homomethyl salicylate as well as mixtures thereof. Most preferably butyl methoxydibenzoylmethane, octocrylene, 4-methylbenzylidene camphor and homosalate are present. Preferably, butyl methoxydibenzoylmethane is used in an amount ranging from 1 to 5 wt.-%, 4-methylbenzylidene camphor in an amount of 1 to 5 wt.-%, octocrylene in an amount of 2 to 10 wt and homosalate in an amount of 1 to 10, based on the total weight of the composition. Most preferably, the sun care preparations are O/W emulsions comprising an oily phase dispersed in an aqueous phase in the presence of an O/W emulsifier and the O/W emulsifier is potassium cetylphosphate, preferably used in an amount ranging from 1 to 5 wt.-%.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention, are e.g. described in the CTFA Cosmetic Ingredient Handbook, Second Edition (1992) without being limited thereto.

The necessary amounts of the cosmetic and dermatological adjuvants and additives can based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The following examples are provided to further illustrate the compositions and effects of the present invention. These examples are illustrative only and are not intended to limit the scope of the invention in any way.

### EXAMPLE

The compositions given in table 1 were prepared according to standard methods in the art.

TABLE 1
Emulsions

| Trade Name | INCI Name | 1 | 2 (Ref) | 3 (Ref) |
|---|---|---|---|---|
| PARSOL ® 1789 | Butyl Methoxydibenzoylmethane | 4.5 | 4.5 | 4.5 |
| PARSOL ® 340 | Octocrylene | 8 | 8 | 8 |
| AMPHISOL ® K | Potassium Cetyl Phosphate | 2 | 2 | 2 |
| Lanette O | Cetearyl Alcohol | 1.5 | 1.5 | 1.5 |
| Mixed Tocopherol 95 | Tocopherol | 0.2 | 0.2 | 0.2 |
| Antaron V-216 | VP/Hexadecene Copolymer | 1 | 1 | 1 |
| PARSOL ® 5000 | 4-Methylbenzylidene Camphor | 3 | 3 | 3 |
| PARSOL ® HMS | Homosalate | 5 | 5 | 5 |
| PARSOL ® TX | Titantium Dioxide & Dimethicone & Silica | 3 | 3 | 3 |
| X-Tend 226 | Phenethyl Benzoate | 5 | 5 | |
| Tinogard ® TL | Benzotriazolyl Dodecyl p-Cresol | 5 | | 5 |
| Cetiol CC | Dicaprylyl carbonate | | 5 | 5 |
| 1,3-Butylenglykol | Butylene Glycol | 3 | 3 | 3 |
| Edeta BD | Disodium EDTA | 0.1 | 0.1 | 0.1 |
| Pemulen ® TR-1 | Acrylates/C10-30 Alkyl Acrylate | 0.15 | 0.15 | 0.15 |
| | Crosspolymer | | | |
| Water dem. | Aqua | 51.1 | 51.1 | 51.1 |
| Keltrol ® CG SF | Xanthan Gum | 0.15 | 0.15 | 0.15 |
| Trizma Base 25% Losung | Tromethamine | 1.3 | 1.3 | 1.3 |
| Ethanol | Alcohol | 6 | 6 | 6 |
| Average whitening | | 2 | 1.5 | 1.6 |

The whitening effect of the compositions was assessed using a panel of six persons. Each person applied 0.09 g of each of the respective emulsion on the forearm on marked circles of a diameter of 5 cm. Afterwards the whitening effect of the different samples was assessed visually and rated against each other with marks from 1 (no whitening) to 3 (significant whitening).

As can be retrieved from the table, the combination of a phosphate ester emulsifier, a benzotriazole derivative of formula (I), Phenylethyl Benzoate and a Titanium dioxide leads to an increased whitening effect of the skin.

1. A topical composition comprising a phosphate ester emulsifier, a cosmetic solvent selected from the group of benzoate solvents, a microfine metal oxide particle and at least one benzotriazol derivative of formula (I) 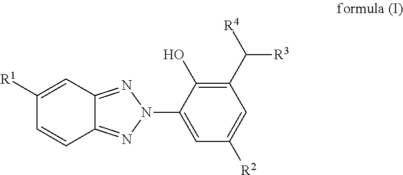
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloallwl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.

2. A topical composition according to claim 1, characterized in that the benzoate solvent is phenethyl benzoate.

3. A topical composition according to claim 1, characterized in that the benzotriazol derivative of formula (I) is present in an amount ranging from 1 to 20 wt.-% based on the total weight of the composition.

4. A topical composition according to claim 1, characterized in that the benzotriazol derivative is used in an amount selected in the range of 2 to 15 wt.-% based on the total weight of the composition.

5. A topical composition according to claim 1, characterized in that the microfine titanium dioxide is used in an amount selected in the range of 1 to 50 wt.-% based on the total weight of the composition.

6. A topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

7. A topical composition according to claim 1, characterized in that the microfine metal oxide particle is microfine titanium dioxide.

8. The topical composition according to claim 7 characterized in that the titanium dioxide is a double coated titanium dioxide having an inner inorganic silica coating and an outer organic coating selected from silicone oils, alkyl silanes, olefinic acids, stearic acid, polyols and organophosphonic acids as well as mixtures thereof.

9. The topical compositions according to claim 8, wherein the outer coating is selected from simethicone, methicone, dimethicone, polysilicone-15, stearic acid, glycerol and mixtures thereof.

10. The topical compositions according to claim 8, wherein the outer coating is selected from methicone and/or dimethicone.

11. A topical composition according to claim 1, characterized in that the phosphate ester emulsifier is a cetyl phosphate.

12. The topical composition according to claim 11, characterized in that the cetyl phosphate is potassium cetyl phosphate.

13. The topical composition according to claim 1, characterized in that the composition comprises at least one co-surfactant in an amount selected in the range of 0.1 to 10 wt.-% based on the total weight of the composition.

14. The topical composition according to claim 13, characterized in that the co-surfactant is selected from the group consisting of cetyl alcohol, cetearyl alcohol, stearyl alcohol, behenyl alcohol, glyceryl stearate, glyceryl myristate, hydrogenated coco-glycerides and mixtures thereof.

15. Use a phosphate ester emulsifier, a solvent selected from the group of benzoate solvents, preferably phenethyl benzoate, and a benzotriazol derivative of formula (I) 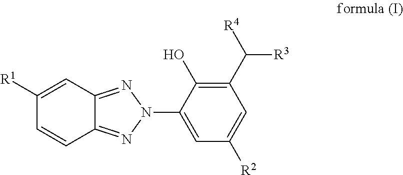
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen;
to increase the whitening effect of the microfine metal oxide particles.

16. Method for increasing the whitening effect of microfine metal oxide particles incorporated into a topical composition said method comprising the addition of a phosphate ester emulsifier, a solvent selected from the group of benzoate solvents, preferably phenethyl benzoate, and at least one benzotriazol of formula (I) 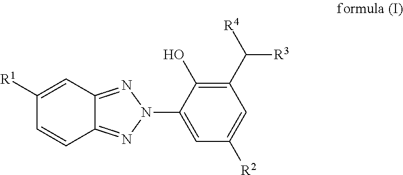
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen;
into said composition and observing or appreciating the result.
